(19) United States
(12) Patent Application Publication
(10) Pub. No.: US 2005/0283046 A1
(43) Pub. Date: Dec. 22, 2005

(54) Method for performing percutaneous lumbar sympathectomy

(76) Inventor: Stephen T. Pyles, Ocala, FL (US)

Correspondence Address: Michelle E. Kandcer;GARDNER GROFF, P.C., 100 Parkwood Point, 2018 Powers Ferry Road, Suite 800, Atlanta, GA, 30339, US

(21) Appl. No.: 11/158,316

(22) Filed: Jun. 21, 2005

Related U.S. Application Data
(60) Provisional application No. 60/581,530, filed on Jun. 21, 2004.

(51) Int. Cl. A61B 1/00

(52) U.S. Cl. 600/102

(57) ABSTRACT

A method for performing a percutaneous lumbar sympathectmony includes the steps of placing the patient in a prone position, monitoring the temperature over each of the patient's lower extremities, making a small incision on the back of the patient, inserting a scope through the incision and into the epidural space, using fluoroscopy to aid in the guidance of the scope to the area of the sympathetic ganglion, locating the sympathetic ganglion, and severing the sympathetic ganglion with, for example, a sharp dissection tool, biopsy forceps, or an electrocautery tool. Thus, the method for performing a percutaneous lumbar sympathectomy is a minimally invasive procedure that minimizes patient recovery time.

### CROSS REFERENCE TO RELATED APPLICATIONS

This application claims priority to U.S. Provisional Patent Application Ser. No. 60/581,530, filed Jun. 21, 2004, which is hereby incorporated herein by reference in its entirety for all purposes.

### TECHNICAL FIELD

The present invention relates generally to the field of surgical procedures and devices, and more particularly to a method and device for performing a percutaneous lumbar sympathectomy.

### BACKGROUND OF THE INVENTION

A typical lumbar sympathectomy is a major operation that is done to increase blood flow and to alleviate long-term pain caused by certain diseases that cause narrowed blood vessels. Typically, a surgeon or a neurosurgeon makes a large incision in the patient's abdomen, and then locates and severs the sympathetic ganglion, which runs parallel to and on either side of the patient's spinal cord. Thus, because of the invasiveness of the surgery, the patient typically has a prolonged recovery time, as well as a long abdominal scar.

Therefore, there exists in the practice a need for a less invasive procedure for performing lumbar sympathectomies that reduces the length of the patient's recovery time and that minimizes the patient's scarring.

### SUMMARY OF THE INVENTION

The present invention provides a method for performing percutaneous lumbar sympathectomies, which is minimally invasive and which minimizes patient recovery time. The method comprises the steps of placing the patient in a prone position, monitoring the temperature over each of the patient's lower extremities, making a small incision in the back of the patient, inserting a scope through the incision and advancing the scope to the anterior lateral surface of the lumbar vertebral body, locating the sympathetic ganglion, and severing the sympathetic ganglion with, for example, a sharp dissection, biopsy forceps, or electrocautery. Preferably, the step of locating the sympathetic ganglion further comprises insufflating carbon dioxide to distend soft tissue from the periosteum over the vertebral body. Also, preferably, the step of advancing the scope further comprises advancing the scope by using fluoroscopic guidance.

The method can comprise the additional steps of using fluoroscopy to identify a landmark so as to identify the location of the incision, sterilizing the skin surface where it corresponds to the landmark, anesthetizing the skin surface where it corresponds to the landmark, taking a picture of the sympathetic ganglion, and/or taking a second picture of the ganglion after it has been severed. In addition, a biopsy of the severed ganglion can be taken and sent to pathology.

In another aspect, the invention includes a kit for performing a percutaneous lumbar sympathectomy, including an antiseptic, a sterile drape, a local anesthetic, a surgical blade, an introducer, a sharp dissection tool, biopsy forceps, or an electrocauterizer, a skin stitch or steri-strip, and optionally a fiberoptic scope, wherein all of the above are packaged in a single kit.

In still another aspect, the invention includes an apparatus for performing a percutaneous lumbar sympathectomy. The apparatus includes a scope, at least one of a sharp dissection tool, biopsy forceps, or electrocautery tool, and/or a surgical blade. Optionally, the apparatus includes a thermometer, a device to measure blood flow, and a carbon dioxide line.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWING

FIG. 1 depicts a flowchart representation of a method for performing a percutaneous lumbar sympathectomy according to an example embodiment of the present invention.

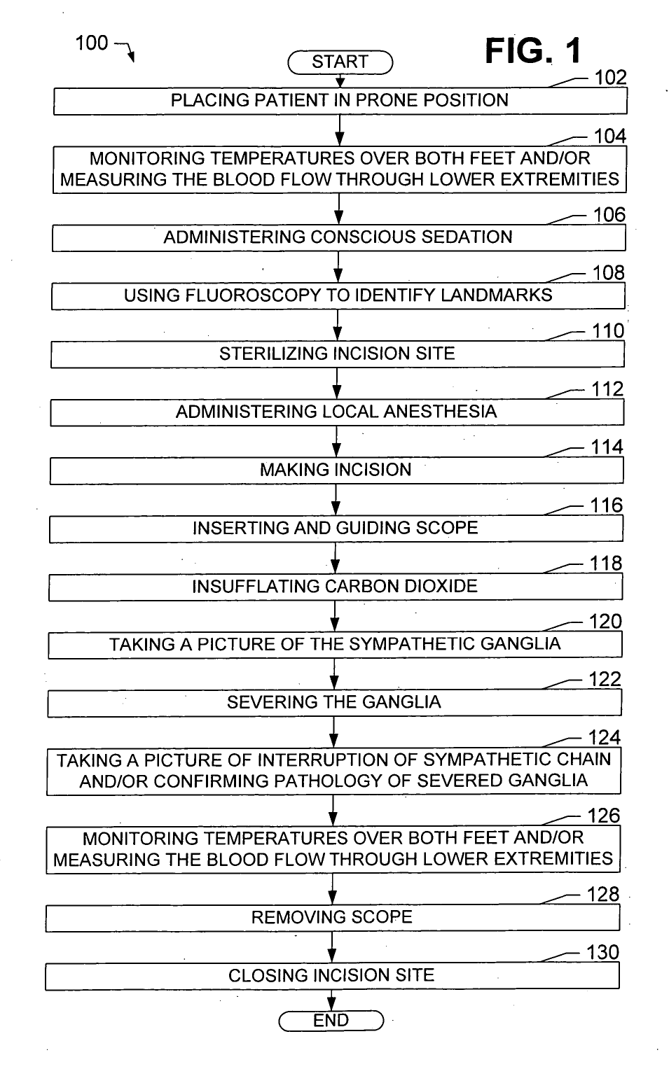

### DETAILED DESCRIPTION OF EXAMPLE EMBODIMENTS

The present invention may be understood more readily by reference to the following detailed description of the invention taken in connection with the accompanying drawing figure, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

A method 100 for performing a percutaneous lumbar sympathectomy is described herein by way of an example embodiment. Beginning at step 102, the practitioner places the patient in a prone position, and monitors the temperatures and/or measures the blood flow over the dorsum of each foot of the patient at step 104, and records a baseline reading. Next, the practitioner, at step 106, administers conscious sedation, using for example, opioid and benzodiazepine.

Then, at step 108, the practitioner uses fluoroscopy, or another imaging technique, to identify landmarks, which correspond to the general location of where the sympathetic ganglion (or ganglia) is to be severed. Anatomical studies have shown that a sympathetic ganglion is predictably located at about the superior anterior lateral surface of vertebra L3. Thus, in an example embodiment, the practitioner can use fluoroscopy to identify an oblique view of the vertebral column at vertebra L3 and the superior anterior lateral surface. Then, the practitioner marks the skin surface with a skin marker at the corresponding location.

At step 110, the site where the skin surface is marked is then sterilized. For example, the practitioner applies an antiseptic solution to the skin surface and then covers the skin surface with a sterile drape having a small opening therein for access to the site to be incised. Next, at step 112, the practitioner applies a local anesthetic, such as for example, 0.5% marcaine with epinephrine, to the surgical tract. Then, at step 114, the practitioner makes a small incision, or stab wound, in the skin, with preferably a #15 surgical blade. Preferably, the incision is on the order of about one-half of a centimeter in length.

Next, at step 116, the practitioner inserts a rigid introducer sheath and/or fiberoptic scope into the incision, through soft tissue and/or muscles, and against the vertebral column at an oblique angle of about 30° to about 45° and uses fluoroscopic guidance until the periosteal surface along the superior anterior lateral surface of L3 is contacted and visualized.

At step 118, the practitioner inserts a carbon dioxide line through the introducer sheath and insufflates carbon dioxide to distend the soft tissue from the periosteum while simultaneously looking for the sympathetic ganglion and/or chain using the fiberoptic scope. The practitioner preferably can move the scope at right angles to the vertebral column so as to locate the sympathetic ganglion. Once the practitioner locates the sympathetic ganglion and/or chain, he takes a picture of it at step 120 by using a camera located at or near the end of the scope. Alternatively, the entire procedure is recorded using video recording equipment coupled to the scope.

Then, the practitioner severs the ganglion and/or chain by using, for example, a sharp dissection tool, biopsy forceps, or electrocautery tool at step 122. After severing the ganglion, the practitioner then takes a picture of the site demonstrating the interruption of the sympathetic chain at step 124 by using the camera located at or near the end of the scope. Additionally, the practitioner can biopsy the severed ganglion and can send a sample of the tissue to pathology for verification that the tissue is in fact from the ganglion or sympathetic chain.

At step 126, the practitioner monitors the temperatures over both feet and/or measures the blood flow through the lower extremities to determine the efficacy of the procedure. Preferably, if the procedure is successful, the flow of blood increases over the dorsum of the patient's feet, which consequently causes the temperatures over the dorsum to increase.

At step 128, the practitioner removes the fiberoptic scope, the carbon dioxide line, and the introducer sheath from the epidural space. Once removed, the practitioner closes the incision with, for example, a skin stitch or a steri-strip at step 130.

The patient can then be transported to a post anesthesia or surgical care unit for monitoring, and the temperatures over the dorsum of each foot are reassessed prior to discharge. Preferably, the surgical procedure lasts for less than about 30 minutes, and the entire method lasts for about two hours, including the time in the post anesthesia care unit. The patient will preferably receive follow-up care in approximately one week.

In an example embodiment, a tool for carrying out a method of the present invention includes a rigid scope, preferably with a stylet for steerability. Preferably, the scope includes a camera and at least one of a sharp dissection tool, biopsy forceps, or electrocautery tool. Alternatively, the scope can be adjustable or flexible. In such case, a needle, a guidewire, and a sheath will be such that the needle will be inserted into the location where the scope will be placed. The guidewire is placed through the needle, and the needle is removed leaving the guidewire in place. The sheath is placed over the guidewire, and the guidewire is removed. The flexible scope can be inserted through the sheath and into the area of the sympathetic ganglion. The tool can optionally include a skin marker for marking the area of the skin to be incised. Also optionally, the tool can include a surgical blade for incising the skin. Other optional components include a thermometer, a device to measure blood flow, and a carbon dioxide line coupling.

The present invention also includes a kit for performing a percutaneous lumbar sympathectomy. The kit can include one or more of an antiseptic; a sterile drape; a local anesthetic; a surgical blade; an introducer; a sharp dissection tool, biopsy forceps, or an electrocauterizer; a skin stitch or steri-strip; a skin marker; and/or a fiberoptic scope having a camera or camera coupling on an end thereof. Preferably, all of the components of the kit are packaged together.

While the invention has been described with reference to preferred and example embodiments, it will be understood by those skilled in the art that a variety of modifications, additions and deletions are within the scope of the invention, as defined by the following claims.

1. A method for performing a percutaneous lumbar sympathectomy, comprising:
placing the patient in a prone position;
making an incision in the skin surface at a location on the back of the patient;
inserting a scope through the incision;
advancing the scope until it is positioned at a desired location;
locating a sympathetic ganglion; and
severing the ganglion.

2. The method of claim 1, wherein the step of locating the sympathetic ganglion further comprises insufflating carbon dioxide to distend soft tissue from the periosteum.

3. The method of claim 1, wherein the step of advancing the scope further comprises advancing the scope by using fluoroscopic guidance.

4. The method of claim 1, further comprising the step of using fluoroscopy to identify a landmark so as to identify the location of the incision.

5. The method of claim 4, wherein the landmark is vertebra L3.

6. The method of claim 4, further comprising the step of sterilizing the skin surface where it corresponds to the landmark.

7. The method of claim 1, further comprising the step of anesthetizing the skin surface where it corresponds to the landmark.

8. The method of claim 1, further comprising the step of recording an image of the sympathetic ganglion.

9. The method of claim 8, further comprising the step of recording a second image of the ganglion after it has been severed.

10. The method of claim 1, further comprising the step of biopsying the severed ganglion and confirming the pathology of the sample.

11. The method of claim 1, further comprising measuring the blood flow through the patient's lower extremities.

12. The method of claim 1, further comprising monitoring the temperature over the lower extremities.

13. The method of claim 1, wherein making an incision in the skin surface at a location on the back of the patient includes making an incision about the area of the superior anterior lateral surface of vertebra L3.

14. A kit for performing a percutaneous lumbar sympathectomy, comprising:
an antiseptic;
a sterile drape;
a local anesthetic;
a surgical blade;
an introducer;
a sharp dissection tool, biopsy forceps, or an electrocauterizer; and
a skin stitch or steri-strip, wherein all of the above are packaged in a single kit.

15. The kit of claim 14, and further comprising a fiberoptic scope.

16. An apparatus for performing a percutaneous lumbar sympathectomy, comprising:
a scope;
at least one of a sharp dissection tool, biopsy forceps, or electrocautery tool; and
a surgical blade.

17. The apparatus of claim 16, wherein the scope is rigid.

18. The apparatus of claim 16, wherein the scope is flexible.

19. The apparatus of claim 16, further comprising a thermometer and/or a device to measure blood flow.

20. The apparatus of claim 16, further comprising a carbon dioxide line.
